(19) United States
(12) Patent Application Publication
(10) Pub. No.: US 2005/0124008 A1
(43) Pub. Date: Jun. 9, 2005

(54) Homogeneous competition assays

(76) Inventor: Lawrence M. Kauvar, San Francisco, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 11/003,675

(22) Filed: Dec. 3, 2004

Related U.S. Application Data
(60) Provisional application No. 60/527,127, filed on Dec. 5, 2003.

(51) Int. Cl. C12Q 1/68; G01N 33/53; G01N 33/542

(52) U.S. Cl. 435/7.9; 435/6

(57) ABSTRACT

Homogeneous assays wherein an unlabeled analyte is detected by displacing a more weakly binding tracer from a binding partner are described. The tracer has signal generating properties which differ when bound or unbound to the binding partner.

This application claims priority under 35 U.S.C. § 119 from provisional application Ser. No. 60/527,127 filed 5 Dec. 2003. The entire contents of this document is incorporated herein by reference.

### TECHNICAL FIELD

The invention relates to methods for determining the presence or concentration of an analyte in a homogeneous assay which can be adapted to intracellular applications. More specifically, the invention concerns detecting the inherent alteration of the signal from a labeled compound displaced from its bound condition by a more strongly bound analyte.

### BACKGROUND ART

Homogeneous assays are those in which the analyte is measured without a physical separation step, making this class particularly useful for high throughput use. A number of homogeneous assay formats are already known in the art. For example, formation of turbidity is used as a measure of agglutination in assays such as those disclosed in U.S. Pat. Nos. 5,589,401 and 6,274,325. In a related assay, which however does not rely on the formation of macroscopic particles, aggregation mediated by antibodies results in steric occlusion of an enzyme as described in U.S. Pat. No. 5,447,846. Other indicators of interaction include release of dye by a liposome mediated by antibody and complement as set forth in U.S. Pat. No. 4,971,916. Other ways to detect binding or proximity include elicitation of light emission by scintillation when a radioactive nuclide is brought into proximity with a scintillator, quenching of fluorescence by proximity to a solid supported as described in U.S. Pat. No. 4,318,707 and various fluorescence depolarization assays which take advantage of the slower decay time of polarized light emitted by a population of materials when they are part of a larger, and thus more lethargic, complex. Other assays which rely on proximity of a label relative to a light source are described in U.S. Pat. Nos. 6,340,598; 5,578,499; and 5,919,712.

Another class of assays rely on manipulation of enzyme active sites, for example, the cloned enzyme-donor immunoassay (CEDIA®) is described in U.S. Pat. No. 5,643,734. In this assay, an analyte is coupled to a portion of an enzyme designated an “enzyme donor,” and allowed to interact with an “enzyme acceptor” which comprises the remaining portions of the enzyme that are required for activity. In the absence of an interfering analyte-binding protein, such as an antibody, this interaction occurs spontaneously and the enzyme reconstitutes itself and exhibits activity. However, when analyte-binding protein is present, this reconstitution is prevented. The amount of free analyte in solution can thus be measured by virtue of the ability of the analyte to compete for the analyte-binding protein thus permitting reconstitution of the enzyme.

A further type of homogeneous assay is particularly relevant. As described by Epoch Bioscience, Lukhtanov, E. A., et al., Am. Biotech. Lab. (2001) 19:68-69, a DNA probe is constructed which folds onto itself in such a manner as to bring a fluor at the 5′ end into close proximity to a quencher at the 3′ end. When the probe hybridizes to an exogenous DNA, the intramolecular structure is disrupted and the proximity of the fluor and quencher is eliminated, resulting in stimulation of fluorescence.

Thus, in principle, there is a multiplicity of homogeneous assays available in the art.

There also exist assays which can be conducted intracellularly. For example, the activity of calcium ion channels is assessed by modifying cells to contain dyes that fluoresce upon binding calcium. Voltage dependent dyes are available to monitor changes in membrane potential. U.S. Pat. No. 6,037,137 describes an assay wherein peptides are provided with two fluorophores one of which quenches the emission of fluorescence from the other; these modified peptides can be used to monitor the activity of protease as cleavage abolishes the quenching. Further, green fluorescent protein (GFP) has been fused to translocating proteins wherein the translocation is monitored in adherent cells using evanescent wave technology to visualize the GFP only when it translates to the basal cell membrane attached to the glass slide.

The present invention provides a competitive assay which relies on displacement of a labeled substance by an analyte, wherein each molecule of the label changes an intrinsic detectable characteristic when displaced, thus avoiding the need for the label to interact with anything other than the ambient solvent in order to be detected. For example, if tracer fluorescence is quenched in the binding site of an antibody, then displacement will increase the fluorescence of each displaced molecule. A further aspect of the invention distinguishes the present method from prior competitive assays, such as those known for the purpose of screening compounds for their ability to bind to a binding moiety that is labeled with its specific binding partner. In these assays, a high affinity labeled tracer for the binding moiety is normally supplied, typically being an analog of the known ligand for which a novel competitor is sought; for example, [125-I]-insulin is a well known tracer for the insulin receptor. With such a tracer/receptor pair, those compounds from a large library that bind to the receptor may be identified by virtue of their ability to displace the ligand known to bind the receptor. The affinity of the library compound is typically lower than the tracer, with high concentrations of compound displacing low concentrations of labeled tracer. In the present invention, however, the affinity of the interaction between binding moiety and the analyte is at least one order of magnitude greater than the affinity of the binding moiety for the tracer. For this purpose, the labeled tracer need not have any obvious structural similarity to the analyte. The feasibility of assays based on this principle is established by U.S. Pat. Nos. 5,338,659; 5,674,688; 5,356,784; and 5,620,901, which establish that proteins bind small organic molecules of diverse structure across a large range of affinities. When such a relatively low affinity label has the property that displacement changes an intrinsic property of the molecule, it becomes feasible to assay analytes even in an intracellular environment.

### DISCLOSURE OF THE INVENTION

The invention is grounded on the understanding that an analyte, known to bind strongly to its binding partner will displace a labeled counterpart which has been bound with weaker affinity to the binding partner for the analyte. Thus, the method of the invention requires the availability of a tracer substance which will bind to a binding partner for the desired analyte with an affinity that is 10-100 times weaker than the affinity of the analyte. Under these conditions, the weaker binding tracer compound, previously bound to the binding partner, will be displaced in proportion to the concentration of analyte present.

Thus, in one aspect, the invention is directed to a method to determine the presence or concentration of an analyte which method comprises contacting a sample in which analyte presence or concentration is to be determined with a binding partner for said analyte wherein said binding partner is bound to a labeled tracer which provides an intrinsically detectable signal when free of said binding partner which is different from its signal when bound to the binding partner and wherein said labeled tracer binds the binding partner with less affinity than does the analyte, and detecting or measuring any change in the signal from the label.

In a particularly preferred embodiment, the sample is the intracellular compartment.

The invention is also directed to kits for performing the assay methods of the invention. The kits will contain a binding partner for the analyte as well as a tracer whose affinity for the binding partner is at least 10-fold less than the affinity of the analyte for the binding partner. The tracer will also have the property of exhibiting an alteration in a detectable characteristic when bound as opposed to unbound to the binding partner.

### BRIEF DESCRIPTION OF THE DRAWINGS

FIG. 1 is a diagrammatic representation of the method of the invention.

### MODES OF CARRYING OUT THE INVENTION

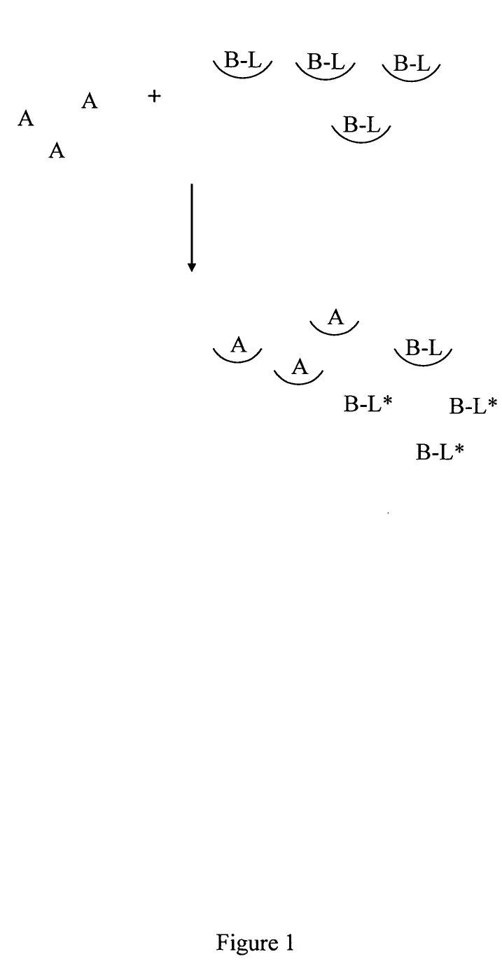

In general, the method of the invention permits the detection or assessment of the concentration of an analyte in a homogeneous format, including performance in an intracellular context. The detection or assessment of concentration relies on the displacement of a bound labeled tracer by the analyte under conditions where the characteristics associated with detecting the label are altered by its displacement. A diagrammatic representation of this concept is shown in FIG. 1 where the analyte, A when contacted with labeled substance B-L bound to another moiety, shown as a semicircle, results in liberating as many labeled B-L units as there are analyte units present in the sample. As indicated by the asterisk (*), the characteristics of the label are altered when it is freed from the binding moiety. In order for the assay to be effective, A must be capable of displacing B-L, which is assured by requiring that the affinity of A for the binding moiety be substantially higher than the affinity of B-L therefor. Typically, the affinity of the analyte for the binding partner or binding moiety will be at least 10 fold, and preferably 100 fold, higher than that of the more weakly bound label. (Of course, the label cannot be so weakly bound that it cannot maintain its status in the sample as substantially bound to the binding moiety in the absence of the analyte.) Thus, if the affinity of the binding moiety for B-L is 10⁻⁷ and for the analyte it is 10⁻⁹, then if B-L is present at 10⁻⁸M (total), ˜99% will be bound in the absence of A assuming an excess of binding moiety sites, but in the presence of 10⁹M A, ˜50% of B-L will be displaced by A, representing a readily measurable change in signal.

As used herein, “binding partner” refers to a substance which can bind both the analyte and the weaker binding label. The binding moiety can, of course, be an antibody or a fragment thereof, including Fab fragments, single chain antibodies (Fv) and the like. It can also be a receptor, a lectin, a carbohydrate, a nucleic acid including nucleic acid aptamers, or any material which preferentially binds analyte but also retains ability to bind to the selected label. For example, if the binding moiety is a nucleic acid, the analyte might be an oligomer that is completely homologous to a portion of the nucleic acid while the label comprises an oligomer which has a lesser degree of homology.

Methods to select pairs of materials with the required binding ratio are available in the art. Since the analyte is known, a binding moiety with a high level of affinity for the analyte can readily be generated by, for example, raising antibodies to the analyte, selecting an appropriate aptamer, or, if the analyte is a ligand or receptor, using the appropriate counterpart or fragment thereof, such as the extracellular binding domain for a hormone. The more weakly binding moiety can then be obtained from combinatorial libraries or other libraries of compounds. Any screening technique, of the many known in the art, can be used. Selection for a defined ratio in binding affinities is readily implemented using the known analyte as a competitor.

As used herein “tracer” refers either to the signal generating moiety itself or to the signal generating moiety already coupled to the substance which is a weaker binder for the binding moiety.

Thus, if the tracer itself has the appropriate binding characteristics for the binding partner as compared to the binding affinity of the analyte and also is capable of generating a detectable signal which is altered when bound as opposed to unbound to the binding partner, such a single moiety may be used. Alternatively, a compound that has the appropriate binding affinity for a binding partner may be linked covalently to a label which has the appropriate properties of signal generation altering in the bound and unbound state.

Signal generating moieties whose characteristics change depending on their environment are known in the art. Many ligands either increase or decrease in intensity of fluorescence depending upon their bound or unbound status and depending upon the nature of the material to which they are bound. For example, Kranz, D. M., et al., Proc. Natl. Acad. Sci. USA (1981) 75:5807-5811 employ the characteristic whereby binding to an antibody quenches fluorescence of fluorescein to monitor immunoglobulin recombination and active site formation. When antibodies containing light chains with IgG1 or IgG2 heavy chains were allowed to immunoreact with fluorescein, the fluorescence of fluorescein was quenched more than 90%. Thus, fluorescence quenching was a practical way to monitor reconstitution and active site formation on mixing resolved heavy and light chains.

Rothstein, T. L., et al., Mol. Immunol. (1983) 20:161-168 used a similar phenomenon, that of fluorescence quenching of p-azophenylarsonate by various anti-idiotypic antibodies, as a measure of affinity of the antibody for the dye. The higher the fluorescence quenching by virtue of the binding of the dye to antibody, the higher the affinity.

Metal-based complexes may also exhibit quenching upon sequestration in organic environments, such as by binding to antibodies or assembled receptors. For example, fluorescence quenching of rubidium complexes occurs upon binding to antibodies raised against such complexes as described by Shreder, K., et al., J. Am. Chem. Soc. (1996) 118:3192-3201. Thus the signal will be enhanced when the labeled compound is freed.

In contrast, the signal may be diminished when the compound is freed, since, as shown by Parker, C. W., et al., Biochemistry (1967) 6:3417-3427, certain antibodies raised against dansyl lysine effect a 150-fold enhancement of the fluorescence of dansyl lysine when the fluorescent compound is bound to the antibody.

Other examples of instances where fluorescence is altered by binding to an organic molecule, such as an antibody, include the binding of “Quantum Dots” which are clusters of metal atoms. Their fluorescence properties are greatly enhanced and tuned to a narrow emission frequency by appropriate molecular environments as described in U.S. Pat. No. 6,207,392 and by Bruchez, Jr., M., et al., Science (1998) 281:2013-2016.

Similarly, the NMR properties of compounds are often influenced by their environment, specifically their ability to interact with water molecules. For example, an assay has been described which is based on enzymatic cleavage of a gadolinium containing compound to expose the metal to water, thereby drastically changing the NMR spectrum (Angew. Chem. Int. Ed. Engl. 36:726 (1997)).

The following examples are intended to illustrate but not to limit the invention.

### EXAMPLE 1

### Measurement of Nucleic Acid Analyte

A single-stranded nucleic acid probe coupled to a fluorescence donor at its 3′ end is prepared and associated with a mismatched tracer coupled to a fluorescence acceptor at its 5′ end. Energy transfer thus occurs when the probe and tracer are in close proximity, and fluorescence is quenched, or the emission wavelength is shifted. Analyte, which is the single-stranded complement to the probe, is measured by contacting a sample containing analyte with the tracer-associated probe and detecting enhancement of fluorescence. This assay may be employed to measure production of mRNA or viral DNA replication in vitro or intracellularly, as single-stranded nucleic acid is generated in these processes.

### EXAMPLE 2

### Intracellular Detection of Antigen

Toxoplasma gondii infects most mammalian cells, undergoing a poorly understood transition from a rapidly replicating form to a dormant form, which is widely present in humans and their companion animals (including both pets and pests). The initially replicating form triggers an immune response and is normally not a serious threat. However, reactivation of replication poses serious risks to newborns and to immunocompromised individuals. Thus, it is desirable to prevent transition to the dormant form and/or to prevent reactivation. (A first draft of the 80 Mb genome sequence of T. gondii has been produced, permitting design of the expression vectors described below.)

The antigen associated with the dormant form is assayed intracellularly to study the genetic control of this transition and for drug screening. A set of high affinity antibodies to the antigen is prepared and screened for the ability to bind to a peptide derived from the antigen more weakly than to the protein. The identified peptide is coupled to a fluorophore which is quenched when bound to the antibody to obtain a tracer. The antibody and tracer are introduced into cells by any method known in the art. Production of the dormant stage antigen is thus measured in single cells by release of the tracer and enhancement of fluorescence using microscopic detection.

Fragments of the genome are cloned into expression vectors and tested as above for their ability to induce the transition, and compounds are screened for their ability to suppress this transition.

### EXAMPLE 3

### Detection of Tyrosine Phosphorylation

A set of antibodies is prepared that bind a tyrosine-containing peptide coupled to a fluorophore (the tracer). A further selection step, including mutagenesis if needed, identifies among that set an antibody that binds to one of the phosphotyrosine (pY) containing sequences in the activated insulin receptor intracellular domain at higher affinity than to the fluorescent tracer. Upon insulin receptor activation, the site becomes phosphorylated. As it has higher affinity for the antibody than does the tracer, the tracer is displaced, resulting in enhanced fluorescence. Thus, a competitive immunoassay for insulin receptor activation is conducted inside the living cell, allowing this important signal transduction step to be visualized in situ.

### EXAMPLE 4

### Detection of Neuronal Activity

Phosphorylated 2-deoxyglucose (2 DG) is formed inside a cell when it takes up 2 DG from the surrounding media. An analog of 2 DG (Nucl Med Biol (1999) 26:833-839) that binds weakly to an antibody for phosphorylated 2 DG is prepared. The analog coupled to fluorophore to form a tracer is freely permeable into cells; the fluorophore is selected so that emission is enhanced by binding to the antibody.

The tracer is applied to brain tissue. Upon uptake and of phosphorylation of 2 DG, the tracer is displaced and fluorescence decreased. This permits determination of neuronal activity in tissue slices in real time, as such activity is correlated with 2-DG uptake. This assay offers advantages over currently used 2 DG technology employing radioactive 2 DG and autoradiography (high resolution but destructive) or PET scanning (low resolution but useable in vivo).

Alternatively, NMR measurements may be used to detect uptake since the NMR signal is changed when tracer is displaced permitting application to intact animals.

The antibody may also be introduced into the assay system by generation from an expression vector in situ allowing its tissue distribution to be restricted, enabling a wide range of assays for neuronal function in a living, awake, animal. For example, expression in the hippocampus can be used to monitor activity in that section of the brain while the animal performs a learning task.

1. A method to assay for the presence or concentration of an analyte which method comprises contacting a sample to be tested for said presence or concentration of analyte with a binding partner for said analyte, wherein said binding partner is coupled to a tracer
wherein the affinity of the tracer for the binding partner is at least 10-fold weaker than the affinity of the analyte for said partner; and
wherein an intrinsically detectable property of the tracer is altered when said tracer is displaced from said binding partner; and
determining the presence or amount of the change in said detectable property of the tracer,
whereby the presence or concentration of the analyte in said sample is determined.

2. The method of claim 1 in which the intrinsic property that changes is fluorescence, or NMR signal.

3. The method of claim 1, which is performed intracellularly in a living cell.

4. A kit for performing the method of claim 1, which kit comprises, in suitable containers, a binding partner for the analyte and a tracer which has an affinity for the binding partner at least 10-fold weaker than that of the analyte and wherein said tracer has an intrinsically detectable property which changes upon displacement from the binding partner, along with instructions for performing the assay.
